(19) United States
(12) Patent Application Publication
(10) Pub. No.: US 2009/0053164 A1
(43) Pub. Date: Feb. 26, 2009

(54) POWDERY PEARLY LUSTRE COMPOSITION, METHOD FOR THE PRODUCTION THEREOF AND USE THEREOF

(76) Inventors: Helga Opper-Linnert, Neuhaus (DE); Michael Becker, Lauf (DE)

Correspondence Address: OSTROLENK FABER GERB & SOFFEN, 1180 AVENUE OF THE AMERICAS, NEW YORK, NY, 100368403, US

(21) Appl. No.: 11/912,251

(22) PCT Filed: Mar. 24, 2006
(86) PCT No.: PCT/EP2006/002712

§ 371 Date: May 15, 2008

(30) Foreign Application Priority Data
Apr. 25, 2005 (DE) 10 2005 019 431.1

(51) Int. Cl. C09D 5/29; A61K 8/89

(52) U.S. Cl. 424/78.03; 523/171

(57) ABSTRACT

The invention relates to non-dust-forming, pulverulent, substantially binder-free pigment compositions, wherein the pigment composition contains the following constituents:
at least 60% by weight of at least one pearl-luster pigment,
from 1 to 15% by weight of a non-ionic monomeric wetting agent and/or non-ionic wetting agent based on polysiloxane, and
from 1 to 39% by weight of a solvent or solvent mixture,
   the amounts of the constituents totaling 100% by weight.

The invention further relates to a method for the preparation of said pigment composition and to the use thereof.

The invention relates to a pulverulent pearl-luster pigment composition, to a method for the preparation of the pulverulent pigment composition, and to the use thereof.

Pearl-luster pigments are ordinarily sold in powder form of. However, the pigment as such has pronounced dust-forming properties, which have a highly undesirable effect when it is incorporated in a printing ink or a paint.

The dust-forming properties of the pearl-luster pigments powder can be reduced if the pearl-luster pigments exist in the form of a pigment composition. The pigment compositions usually contain binder additives and frequently small quantities of solvent.

EP 0 994 166 A1 discloses a non-dust-forming homogeneous pigment composition containing at least 50% by weight of one or more effect pigments, from 0.1 to 50% by weight of a mixture of plasticizer and nitrocellulose, the ratio of plasticizer to nitrocellulose being from 1:1 to 1:9, and from 0 to 49.9% by weight of a solvent or solvent mixture. However, this pigment composition is still dust-forming at very low plasticizer to nitrocellulose concentrations, and at higher plasticizer to nitrocellulose concentrations it is not compatible with water-based application systems.

DE 198 26 624 A1 discloses a non-dust-forming homogeneous pigment composition containing at least 40% by weight of one or more effect pigments, from 0.5 to 60% by weight of a polyalkylene glycol and/or one or more polyalkylene glycol derivatives. According to DE 100 9826 624 A1 the pigment composition preferably contains considerable quantities of binder. Such a pigment composition may also only be incorporated in application systems in which the binder of the application systems and that in the pigment composition are compatible.

DE 102 28 199 A1 discloses pigment compositions in which, besides anionic szrface active additives, also nonionic szrface active additives based on polyethers are present.

DE 102 27 657 A1 discloses solid pigment compositions containing from 60 to 90% by weight of pigment and from 10 to 40% by weight of at least one szrface active additive from the group of nonionic polyethers and their acid phosphoric, phosphonic, sulfuric and/or sulfonic esters and the salts thereof. These pigment compositions are suitable only for coloring plastics.

The disadvantage of these known pigment compositions is the fact that after being used for printing in a water-based printing ink, the resulting dry printed product does not have adequate water resistance.

It is an object of the present invention to provide a concentrated stock form of pearl-luster pigments that is not dust-forming and is flowable and displays maximal compatibility with the application system. In addition, the stock form of the pearl-luster pigments should be printable in aqueous printing ink, and the printed products obtained should have very good water resistance.

This object is achieved by the provision of a non-dust producing, pulverulent, substantially binder-free pigment composition, said pigment composition containing the following constituents:
at least 60% by weight of at least one pearl-luster pigment,
from 1 to 15% by weight of a nonionic monomeric wetting agent and/or nonionic wetting agent based on polysiloxane, and
from 1 to 39% by weight of solvent or solvent mixture,
   the amounts of the individual constituents totaling 100% by weight.

Preferred embodiments are defined in the subordinate claims.

According to a preferred development of the invention, the nonionic wetting agent carries OH groups. The OH group-carrying nonionic wetting agent preferably has a hydroxyl number of from 30 to 150 KOH/g of wetting agent and preferably from 50 to 120 mg KOH/g of wetting agent. A hydroxyl number of from 75 to 110 has been found to be very suitable. The hydroxyl number can be determined as specified in DIN EN ISO 4629.

According to one variant of the invention, the pigment composition additionally contains from 1 to 10% by weight and preferably from 2 to7% by weight of defoamer. The use of defoaming agents may be advantageous if the application medium in which the pigment composition of the invention is incorporated tends to foam or form bubbles.

The pearl-luster pigment used can be any pearl-luster pigment, in particular conventional pearl-luster pigments available on the market.

The pearl-luster pigments are preferably selected from the group consisting of micas coated with highly refractive metal oxide(s), glass platelets coated with highly refractive metal oxide(s), SiO₂ flakes coated with highly refractive metal oxide(s), Al₂O₃ flakes, TiO₂ flakes, iron oxide flakes coated with highly refractive metal oxide(s), and mixtures thereof.

Pearl-luster pigments generally consist of a low-refractive platelet-like substrate and a highly refractive metal oxide or metal sulfide.

The term “pearl-luster substrates” is to be understood to mean preferably mica, kaolin, glass platelets, synthetic SiO₂ flakes such as those obtained according to the teaching of EP 1 266 977 A2, or synthetic aluminum oxide platelets.

The highly refractive metal oxides or metal oxide hydrates or metal suboxides that are used are preferably derived from the metals in the group consisting of titanium, iron, zirconium, zinc, tin, nickel, cobalt, and/or chromium.

The refractive index of the metal oxide layer, in order to produce a good pearl-luster effect, is preferably greater than 1.8, more preferably greater than 2.2, even more preferably greater than 2.4 and most preferably 2.5 or greater.

Pure titanium dioxide and/or pure iron oxide platelets may alternatively be used as pearl-luster pigments.

The following pearl-luster pigments are preferably used:
titanium dioxide-coated and/or iron oxide-coated mica platelets,
titanium dioxide-coated and/or iron oxide-coated glass platelets,
titanium dioxide-coated and/or iron oxide-coated aluminum oxide platelets,
titanium dioxide-coated and/or iron oxide-coated SiO₂ platelets,
bismuth oxychloride platelets,
   and mixtures of all these pearl-luster pigments.

Substrates coated with titanium dioxide and iron oxide (Fe₂O₃ and/or Fe₃O₄) are preferably used. Mica pigments coated with titanium dioxide and/or iron oxide are commercially available, for example, under the trade names Phoenix® (supplied by Eckart GmbH & Co., KG, Furth, Germany) or under the name Iriodin® (supplied by Merck GmbH & Co. KG), Darmstadt. Al₂O₃ flakes coated with titanium dioxide and/or Fe₂O₃ are sold under the trade names Xirallic® and similarly coated SiO₂ flakes under the trade name Colorstream supplied by Merck GmbH & Co KG, Darmstadt, Germany. Multilayered interference pigments such as are described in DE19618 569 consisting of a support material coated with alternating layers of metal oxides with low and high refractive indices may be used according to the invention.

Such pearl-luster pigments intended for external applications are provided with additional protective layers imparting weather stability. Such protective layers contain metal oxides and/or metal hydroxides preferably selected from the group consisting of SiO₂, Al₂O₃, cerium oxide, zirconium oxide, and mixtures thereof.

Those pearl-luster pigments are considered to be platelet-like which have a form factor (ratio of their average length to their average thickness) of from 3 to 2000, preferably from 5 to 500 and more preferably from 10 to 350.

The dimensional ratios of the platelet-like pearl-luster pigments are characterized by an average value (d₅₀ value) of the cumulative size distribution curve—ordinarily measured by laser diffraction methods (e.g. Malvern provided by Malvern, Germany). Preferably, the size has a d₅₀ value of from 1 to 500 μm, more preferably from 3 to 100 μm and very preferably from 5 to 35 μm.

In addition, for the purposes of the invention, the term “pearl-luster pigments” also refers to pigments having an opalescent structure such as is disclosed in the patent applications DE 102 28 228 A1 or WO 01/880 44 A1, or an inverse opalescent structure such as is disclosed in DE 102 45 848 A1. Unlike the conventional pearl-luster pigments, such pigments do not necessarily have a platelet-like structure.

The solvent is preferably water. When a solvent mixture is used, it is preferable for it to consist substantially of water. Such a solvent mixture preferably contains at least 70% by volume of water, more preferably at least 80% by volume of water, and most preferably at least 90% by volume of water. According to another variant, the solvent mixture contains at least 95% by volume of water.

According to one aspect of the present invention, the pigment composition contains less than 1% by weight and preferably less than 0.5% by weight of binders. The pigment composition is preferably free of binder.

A great advantage of the pigment composition of the invention is the extremely low content of binder or the total absence of binder.

The pigment composition of the invention may also very advantageously be implemented in a great variety of application systems without having to expect complications due to incompatibilities of different binders.

According to a preferred variant of the invention, the monomer wetting agent displays the following structure (I):

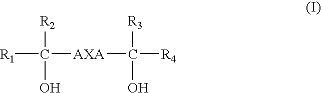

wherein R₁, R₂, R₃, and R₄ independently stand for hydrogen, a straight-chain or branched-chain alkyl radical containing from 1 to 10 carbons and optionally having OH groups, an aryl radical containing from 1 to 12 carbons, which aryl radical may be substituted by alkyl radicals containing from 1 to 10 carbon atoms, each alkyl and/or aryl radical optionally having OH groups, and X stands for a single, double, or triple bond, wherein A stands for CH₂ when X is a single bond, A stands for CH when X is a double bond, and A stands for C when X is a triple bond.

R₁, R₂, R3, and R₄ are independently preferably methyl, ethyl, n-propyl, isopropyl, n-butyl, isobutyl, tert-butyl. R₁ and R₄ are also preferably tert-butyl and R₂ and R₃. Preferably, R₁, R₂, R₃, and R₄ each stand for a methyl radical.

According to another preferred variant, the wetting agent based on polysiloxane is a polysiloxane substituted by alkyl, aryl, alkoxy and/or polyalkylene oxide radicals, said polysiloxane displaying the following structure (II):

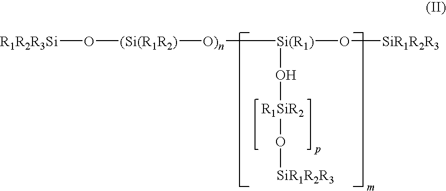

wherein R₁, R₂, and R₃ independently stand for alkyl, aryl, alkoxy and/or polyalkylene oxide radicals and
n stands for an integer from 1 to 1000 and preferably from 5 to 500,
p stands for an integer from 1 to 1000 and preferably from 5 to 500, and
m stands for an integer from 0 to 50 and preferably from 0 to 25.

According to a preferred variant, n and p have the same numerical value.

In the polysiloxane-based binder, the alkyl radicals are preferably independently selected from the group consisting of methyl, ethyl, n-propyl, isopropyl, n-butyl, isobutyl, and tert-butyl. These alkyl radicals are preferably all methyl.

The alkoxy radicals are preferably independently selected from the group consisting of methoxy, ethoxy, n-propoxy, isopropoxy, isobutoxy, and tert. butoxy. The alkoxy radicals are preferably methoxy and/or ethoxy.

The polyalkylene oxide radicals are preferably independently selected from the group consisting of polymethylene oxide, polyethylene oxide, polypropylene oxide, and polybutylene oxide. Polyethylene oxide has been found to be very suitable.

According to a preferred embodiment, the polyalkylene oxide radicals are composed of from 1 to 100, preferably from 2 to 50 and more preferably from 5 to 20 alkylene oxide units.

The polysiloxanes disclosed in U.S. Pat. No. 6,858,663 B2 have also been found to be very useful. The disclosure of U.S. Pat. No. 6,858,663 B2 is incorporated herein by reference.

It has been found, surprisingly, that when nonionic wetting agents as per structure (I) or structure (II) are used, the pearl-luster pigments, which are flat pigments having a length of preferably from approximately 1 μm to 200 μm and more preferably from approximately 10 to 150 μm, and having a thickness preferably of from approximately 0.3 μm to 10 μm and more preferably from approximately 1 μm to approximately 5 μm, show outstanding orientation behavior in an applied paint, printing ink, or enamel. This orientation behavior is very important for the visual impression on an observer, since it very favorably influences the optical properties of the ink or paint film. The pearl-luster pigments of the pearl-luster pigment composition of the invention are oriented, after application, substantially parallel to the substrate surface. A substrate surface may, for example, be printing stock such as paper or a vehicle body to be enameled. Because of the good orientation of the pigments, a very good luster is obtained after application.

The object of the present invention is also achieved by a method for the preparation of a pulverulent, non-dust-forming, substantially binder-free pigment composition according to any one of claims 1 to 16, in which the individual constituents are homogeneously intermixed, preferably gently.

The object of the present invention is also achieved by a method for the preparation of a pulverulent, non-dust-forming, substantially binder-free pigment composition according to anyone of claims 1 to 16, in pigmented coating compositions, especially printing inks, paints, and cosmetic formulations.

It has been found, surprisingly, that the prints obtained after printing with printing inks or the coatings obtained after application of paints or enamels are very stable and resistant to aqueous media or water. This stability is traceable to the pigment composition of the invention present in the paints, printing inks and enamels.

The stability or resistance of the prints to water, also referred to as “water fastness”, is determined as specified in DIN 16524 (November 1965).

The term “pulverulent” means, for the purposes of the invention, that the pearl-luster pigments may also exist in the form of aggregates, e.g., as granules. The pearl-luster pigments in the pigment composition of the invention, however, do not exist in compacted forms such as pellets, sausage-shaped extrudates, beads, briquettes, or tablets such as are disclosed in, say, EP 97/104 851.7, which discloses pearl-luster pigment compositions compacted in the presence of binders. Nevertheless, all the advantages of the pigment compositions in compacted forms, such as freedom from dust, volume shrinkage relative to the powder, non-smearing properties etc. are likewise found in the pigment compositions of the invention.

The pigment composition of the invention is flowable and is also easy to meter. An extremely advantageous feature is the fact that the pigment compositions of the invention can be incorporated in application systems for paints, printing inks and enamels in a simple manner such that the pearl-luster pigments are dispersed homogeneously in the application system.

The pigment compositions of the invention display a powdery to granulated non-dust-forming consistency. The pigment compositions of the invention are usually of irregular shape.

The powder form is advantageous over the paste form, which occurs if the pigment content is too low or the solvent content too high, since powder does not smear during incorporation of the pigments in application systems. This has advantages for the user, since the pigment is easy to meter into the application system, and the devices used to incorporate the pigments in an application system comprising, say, inks, especially printing inks, or paints, are less fouled. Since the pearl-luster pigment compositions of the invention, unlike pure pearl-luster pigment powders, which usually have dust-forming properties, do not generate dust, and they are easier to handle from the production engineering and occupational safety standpoint.

The pigment composition of the invention preferably contains from 60 to 95% by weight, more preferably from 70 to 90% by weight and most preferably from 71 to 85% by weight of pearl-luster pigments.

Below 60% by weight, the pigment composition may not be sufficiently concentrated and will assume a pastelike consistency. Above 95% by weight, the pigment composition begins to generate dust.

The nonionic wetting agent greatly facilitates the incorporation of the pigment powder in the application system and application medium. Anionic wetting agents, conversely, often cause strong foam evolution when a pigment composition is incorporated in an application system. In addition, only a low water resistance is obtained for the printed product or painted article.

It has been found, surprisingly, that when the pigment compositions of the invention are used, the printed products or the paint coatings produced have very good water resistance or water fastness.

Printing ink films not over-coated must have good water resistance, after drying, in many applications (such as wallpaper printing, gift wrapping paper, napkins, folding boxes etc.). In such cases, the printed object again comes into contact with water, and the printing ink or its constituents should not become loosened or detached as a result.

Suitable nonionic wetting agents include, e.g., Disperbyk-107 and Disperbyk-108 supplied by Byk-Chemie GmbH (P.O. Box 100245, D-46462 Wesel, Germany) or Tego Twin 4000 (supplied by Tego Chemie Service GmbH, Goldschmidtstr. 100, D-45127 Essen, Germany) or Surfynol 104, Surfynol 104PA or Surfynol 504 (supplied by Air Products Chemicals Europe B.V., Kanaalweg 15, P.O. Box 3193, NL 3502 GD Utrecht, Netherlands).

The wetting agents are used in their commercially available form. These are usually solutions of the wetting agent in solvents. The content of the wetting agent in the pigment composition of the invention refers to the content of wetting agent without the solvent content.

Many well-known pigment compositions contain binders that are compatible with water. These binders are readily redissolved by water. Printed films that can be produced from such pigment compositions consequently display poor water resistance due to the incorporation of the water-compatible binder in the ink film stemming from the pigment composition. Pigment compositions that contain binders having good compatibility with organic solvents conversely display only a limited compatibility with aqueous application systems.

For these reasons the pigment composition of the invention is substantially free of binder. The term “substantially binder-free” preferably means a content of less than 1% by weight, more preferably less than 0.5% by weight, even more preferably less than 0.3% by weight and most preferably less than 0.1% by weight, based on the total pigment composition. The pigment composition very preferably contains no binder at all.

In the small quantities stated above, binders cause no impairment, or no significant impairment, of the compatibility of the pigment composition of the invention with the application systems or of the water resistance of the application, e.g., a printed product or a painted article.

Binders that may be used in very small quantities are preferably those that can be used for printing inks, e.g., acrylates, nitrocelluloses, polyvinyl butadienes, cellulose butyrates, cellulose acetates, polyurethanes, sulfopolyesters, alkyd resins, polyamides, polyesters, melamine resins, or maleates.

In a preferred embodiment, the pigment composition of the invention also contains at least one defoamer. This ensures better incorporation, especially in aqueous systems, without the formation of air bubbles. The defoamer is used in quantities of from 1 to 10% by weight, preferably from 2 to 7% by weight and more preferably from 3 to 5% by weight, based on the total pigment composition. Below 1% by weight, its effectiveness is too low, while above 10% by weight, no further advantageous effects can be noted.

Basically all conventional defoamer systems are suitable as defoamers. These include, for example, silicone oils, medical white oils, or vegetable oils.

Commercially available defoamers are, for example, BYK-071, BYK-A 530, BYK-060 N, BYK-018, BYK-044, BYK-020, BYK-065, BYK-066N or BYK-067 supplied by Byk-Chemie. Compounds of the product series Agitan® supplied by Münzing Chemie GmbH (Salzstraβe 174, D-74076 Heilbronn, Germany) are also suitable.

Defoamers based on mixtures of mineral oils are also suitable. Commercially available examples thereof are:

BYK-031, BYK-035, BYK-037, BYK-038 BYK-045 supplied by Byk-Chemie, Dapro DF 900, Dapro DF 911, and Dapro DF 1492 supplied by Elementis Specialities p/a Elementis Service Centre N.V., Pegasus Park, De Kleetlaan 12A- P.O.Box 3, B-1831 Diegem, Belgium.

The solvents used are water-compatible solvents and mixtures thereof. These include, for example, alcohols, glycols, ketones, aldehydes, or esters.

However, water or a solvent mixture containing water is preferably used. When a solvent mixture is used, preference is given to one consisting substantially of water. Such a solvent mixture preferably contains at least 70% by volume of water, more preferably at least 80% by volume of water and even more preferably at least 90% by volume of water. According to another variant, the solvent mixture contains at least 95% by volume of water.

A method for the preparation of the pigment composition of the invention consists of intermixing the individual constituents homogeneously, preferably in a gentle manner. The blending can take place in commercially available mixers, kneaders, or a tumbling barrel mixer. It is basically possible for the individual constituents of the pigment composition of the invention to be charged into the mixing apparatus in any order and then blended with each other under the gentlest possible conditions. Gentle treatment is preferred, because the shear-sensitive pearl-luster pigments could otherwise be destroyed.

In a preferred method, the wetting agent and optionally the defoamer are dissolved in the solvent, after which this solution/dispersion is mixed with the pearl-luster pigment in the mixing apparatus. This method ensures optimal homogeneity of the pigment composition.

The pigment compositions of the invention may be used in printing inks, paints, and cosmetic formulations. Suitable printing inks are all known printing inks such as offset printing inks, flexo-printing inks, screen printing inks and intaglio printing inks. The pigment composition of the invention is preferably used in aqueous printing inks and aqueous paints.

Aqueous printing inks are in turn used on wallpapers, folding boxes, gift wrapping paper, or tissues. Conventional printing inks are used, for example, in flexible film packaging or in label printing.

The following examples illustrate the invention without restricting the scope thereof.

### EXAMPLES

### Example 1

5 g of Surfynol 104 (wetting agent supplied by Air Products) and 2 g of Agitan 760 (defoamer) are dissolved in 18 g of water with stirring. This solution is mixed with 75 g of PHOENIX PX 2031 (commercially available pearl-luster powder supplied by Eckart) in a butterfly mixer. A non-dust-forming pearl-luster powder is obtained.

### Example 2

8 g of Surfynol 504 (wetting agent supplied by Air Products) and 5 g of Dapro DF 900 (defoamer) are dissolved in 17 g of water with stirring. This solution is mixed with 70 g of PHOENIX PX 3001 (commercially available pearl-luster powder supplied by Eckart) in a butterfly mixer. A non-dust-forming pearl-luster powder is obtained.

### Example 3

5 g of Surfynol 104PA (wetting agent supplied by Air Products) and 5 g of Byk 052 (defoamer) are dissolved in 20 g of isopropanol with stirring. This solution is mixed with 70 g of PHOENIX PX 1000 (commercially available pearl-luster powder supplied by Eckart) in a butterfly mixer. A non-dust-forming pearl-luster powder is obtained.

### Example 4

2 g of Tego Twin 4000 (wetting agent supplied by Tego) and 2 g of No Foam (defoamer) are dissolved in 16 g of water with stirring. This solution is mixed with 80 g of Iriodin 123 (commercially available pearl-luster powder supplied by Merck) in a butterfly mixer. A non-dust-forming pearl-luster powder is obtained.

### Comparative Example 5

5 g of Surfynol 104 (wetting agent supplied by Air Products) and 2 g of Agitan 760 (defoamer) are dissolved in 18 g of water with stirring. This solution is mixed with 50 g of PHOENIX PX 2031 (commercially available pearl-luster powder supplied by Eckart) in a butterfly mixer. Instead of a powder, there is obtained a dilatant paste.

### Comparative Example 6

5 g of Hydropalat 88 (anionic wetting agent based on a sulfosuccinic acid ester, supplied by Henkel) and 2 g of Agitan 760 (defoamer) are dissolved in 22 g of water with stirring. This solution is mixed with 70 g of PHOENIX PX 3001 (commercially available pearl-luster powder supplied by Eckart) in a butterfly mixer. A non-dust-forming pearl-luster powder is obtained.

### Comparative Example 7

2 g of Byk 333 (wetting agent based on a polyether-modified dimethylpolysiloxane containing no OH functions, supplied by Byk) and 2 g of No Foam are dissolved in 16 g of water with stirring. This solution is mixed with 80 g of Iriodin 123 (commercially available pearl-luster powder supplied by Merck) in a butterfly mixer. A non-dust-forming pearl-luster powder is obtained.

### Comparative Example 8

15 g of Luviskol K 17 (polyvinyl pyrrolidone resin supplied by BASF) and 5 g of Surfynol 104 are dissolved in 20 g of water with stirring. This solution is mixed with 80 g of PHOENIX PX 2301 (commercially available pearl-luster powders supplied by Eckart) in a butterfly mixer. A non-dust-forming pearl-luster powder is obtained.

The pigment compositions described in the examples of the invention and comparative examples listed above were tested in printing applications. In each case a flexo-printing ink based on a commercially available acrylate system was prepared. The pigment compositions of the examples were metered such that a concentration of the pearl-luster pigment of 26% by weight, based on the total printing ink, was obtained. The printing inks were applied with the following devices:

1) Lab-intaglio: Laboratory print using K 303 Multicoater (supplied by Saueressig, Germany)

2) Lab flexo-printing: Laboratory print using Flexiproof 100 (supplied by Erichsen, Germany)

3) Intaglio/flexo-printing machine: industrial prints using the intaglio/flexo-printing machine ROTOVA 300 (supplied RotoColor Co., Switzerland), speed 150 m/min.

The properties (water resistance) of the printed films were not determined until the respective film had completely dried.

The laboratory prints (1,2) were dried overnight at room temperature, while the prints obtained using the intaglio/flexo-printing machine were dried at 80° C. under a stream of hot air at a specified belt speed.

The water resistance was tested as specified in DIN 16 524 Sheet 1.

No significant differences were noted in the properties of the prints produced in the different application systems.

TABLE 1

| | Sample | | |
|---|---|---|---|
| Sample | consistency | Film quality | Water resistance |
| Example 1 | Non-dust-forming | Very good | Satisfies criteria of |
| | pearl luster powder | | DIN 16524 Sheet 1 |
| Example 2 | Non-dust-forming | Very good | Satisfies criteria of |
| | pearl luster powder | | DIN 16524 Sheet 1 |
| Example 3 | Non-dust-forming | Very good | Satisfies criteria of |
| | pearl luster powder | | DIN 16524 Sheet 1 |
| Example 4 | Non-dust-forming | Very good | Satisfies criteria of |
| | pearl luster powder | | DIN 16524 Sheet 1 |
| Compara- | Dilatant paste | Very good | Satisfies criteria of |
| tive | | | DIN 16524 Sheet 1 |
| Example 5 | | | |
| Compara- | Non-dust-forming | Surface defects | Satisfies criteria of |
| tive | pearl luster powder | (strong foam | DIN 16524 Sheet 1 |
| Example 6 | | formation) | |
| Compara- | Non-dust-forming | Surface problems, | Satisfies criteria of |
| tive | pearl luster powder | reduced brilliance | DIN 16524 Sheet 1 |
| Example 7 | | | |
| Compara- | Non-dust-forming | Very good | Very poor water |
| tive | pearl luster powder | | resistance |
| Example 8 | | | |

Table 1 shows that Examples 1 through 4 of the invention produce a non-dust-forming pearl-luster pigment powder. These non-dust-forming pigments are very easy to incorporate in printing inks. The printed films produced using these printing inks are of very good quality and are water resistant.

The pigment composition obtained according to Comparative Example 5 has a low pearl-luster pigment concentration of only 50% by weight. In this case, a pulverulent pigment composition is not formed but rather a pigment having dilatant viscous properties, which is undesirable. It was extremely difficult to incorporate this pigment composition in the printing ink. Such a pigment composition is of no practical use because of the difficulty encountered in incorporating the same.

An anionic wetting agent was used for the preparation of the pigment composition according to Comparative Example 6. Despite the addition of a defoamer, there was undesirable vigorous evolution of foam during the incorporation of this pigment composition in the printing ink. Surface effects on the printed films were also observed, traceable to inadequate pigment wetting.

Very poor water resistance was obtained with the pigment composition according to Comparative Example 8, for which a water soluble resin was used instead of a nonionic wetting agent.

1. A non-dust-forming, pulverulent, substantially binder-free pigment composition, wherein
said pigment composition contains the following constituents:
at least 60% by weight of at least one pearl-luster pigment,
from 1 to 15% by weight of non-ionic monomeric wetting agent and/or non-ionic wetting agent based on polysiloxane, and
from 1 to 39% by weight of a solvent or solvent mixture,
the amounts of the constituents totaling 100% by weight, wherein said nonionic wetting agent carries OH groups and has a hydroxyl value of from 30 to 150 mg KOH/g of wetting agent, preferably from 50 to 120 mg KOH/g of wetting agent.

2. The non-dust-forming, pulverulent pigment composition according to claim 1, wherein
said pigment composition additionally contains from 1 to 10% by weight, preferably from 2 to 7% by weight, of defoaming agent.

3. The non-dust-forming, pulverulent pigment composition according to claim 1, wherein
said pearl-luster pigment is selected from the group consisting of micas coated with highly refractive metal oxide(s), glass platelets coated with highly refractive metal oxide(s), SiO₂ flakes coated with highly refractive metal oxide(s), Al₂O₃ flakes, TiO₂ flakes, and iron oxide flakes coated with highly refractive metal oxide(s), and mixtures thereof.

4. The non-dust-forming, pulverulent pigment composition according to claim 1, wherein
said solvent is water or that said solvent mixture consists substantially of water.

5. The non-dust-forming, pulverulent pigment composition according to claim 1, wherein
the pigment composition contains less than 1% by weight, preferably less than 0.5% by weight, of binding agent.

6. The non-dust-forming, pulverulent pigment composition according to claim 1, wherein
the pigment composition is binder-free.

7. The non-dust-forming, pulverulent pigment composition according to claim 1, wherein
the monomeric wetting agent has the following structure (I): 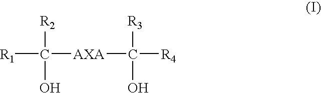
wherein R₁, R₂, R₃, and R₄ independently stand for hydrogen, a straight-chain or branched-chain alkyl group having from 1 to 10 carbons and optionally having OH groups, or an aryl group containing from 1 to 12 carbons, wherein the aryl group can be substituted by alkyl groups containing from 1 to 10 carbons, wherein each of the alkyl and/or aryl groups can have OH groups, and X stands for a single bond, a double bond or a triple bond, wherein A stands for CH₂ when X is a single bond, A stands for CH when X is a double bond, and A stands for C when X is a triple bond.

8. The non-dust-forming, pulverulent pigment composition according to claim 7, wherein
R_{1,} R₂, R₃, and R₄ independently stand for methyl, ethyl, n-propyl, isopropyl, n-butyl, isobutyl, or tert-butyl.

9. The non-dust-forming, pulverulent pigment composition according to claim 7, wherein
R₁ and R₄ each denote tert-butyl and R₂ and R₃ preferably each denote methyl.

10. The non-dust-forming, pulverulent pigment composition according to claim 1, wherein
the wetting agent based on polysiloxane is a polysiloxane substituted by an alkyl, aryl, alkoxy, and/or polyalkylene oxide radical, wherein the polysiloxane has the following structure (II): 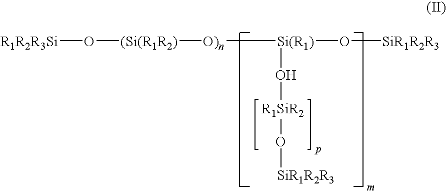
wherein
R₁, R₂, and R₃ independently stand for an alkyl, aryl, alkoxy, and/or polyalkylene oxide radical,
n stands for an integer from 1 to 1000, preferably from 5 to 500,
p stands for an integer from 1 to 1000, preferably from 5 to 500, and
m stands for an integer from 0 to 50, preferably from 0 to 25.

11. The non-dust-forming, pulverulent pigment composition according to claim 10, wherein
the alkyl groups are independently selected from the group consisting of methyl, ethyl, n-propyl, isopropyl, n-butyl, isobutyl, and tert-butyl, and are preferably each methyl.

12. The non-dust-forming, pulverulent pigment composition according to claim 10, wherein
the alkoxy radicals are independently selected from the group consisting of methoxy, ethoxy, n-propoxy, isopropoxy, n-butoxy, isobutoxy, and tert-butoxy, and are preferably each methoxy and/or ethoxy.

13. The non-dust-forming, pulverulent pigment composition according to claim 10, wherein
the polyalkylene oxide radicals are independently selected from the group consisting of polymethylene oxide, polyethylene oxide, polypropylene oxide, and polybutylene oxide and are preferably each polyethylene oxide.

14. The non-dust-forming, pulverulent pigment composition according to claim 10, wherein
the polyalkylene oxide radicals are composed of from 1 to 100, preferably from 2 to 50 and more preferably from 5 to 20 alkylene oxide units.

15. A method for the preparation of a non-dust-forming, pulverulent, substantially binder-free pigment composition according to claim 1, wherein
the individual constituents are homogeneously intermixed, preferably in a gentle manner.

16. The use of the non-dust-forming, pulverulent, substantially binder-free pigment composition according to claim 1 in printing inks, paints, and cosmetic formulations.
